(19) United States
(12) Patent Application Publication
(10) Pub. No.: US 2012/0141384 A1
(43) Pub. Date: Jun. 7, 2012

(54) ANTIBACTERIAL CONJUGATED BORONIC ACIDS AND PHARMACEUTICAL COMPOSITIONS THEREOF

(76) Inventor: Dov Tamarkin, Maccabim (IL)

(21) Appl. No.: 12/435,796

(22) Filed: May 5, 2009

Related U.S. Application Data
(60) Provisional application No. 61/050,767, filed on May 6, 2008.

(51) Int. Cl. A61K 31/69; A61K 9/12; A61P 17/10; A61P 31/04; A61P 17/00

(52) U.S. Cl. 424/45; 514/64

(57) ABSTRACT

A method of treating a disorder associated with a bacterial infection, consisting of administrating a pharmaceutical composition, comprising (a) a therapeutically effective amount of a conjugated boronic acid or a derivative thereof; and (b) a suitable pharmaceutical vehicle. The invention further relates to suitable pharmaceutical vehicles and pharmaceutical compositions for treating such disorders.

### CROSS-REFERENCE TO RELATED APPLICATIONS

This application claims priority under 35 U.S.C. §120 to U.S. Application Ser. No. 61/050,767, filed on May 6, 2008, which is herein incorporated by reference in its entirety.

### BACKGROUND

This application relates to the use of certain conjugated boronic acid compounds against microbiological species, other than fungi, yeasts or molds.

A boronic acid is an alkyl or aryl substituted boric acid containing a carbon to boron chemical bond belonging to the larger class of organoboranes. Boronic acids act as Lewis acids. Their unique feature is that they are capable of forming reversible covalent complexes with sugars, amino acids, hydroxamic acids, etc. (molecules with vicinal, (1,2) or occasionally (1,3) substituted Lewis base donors (alcohol, amine, carboxylate). They are occasionally used in the area of molecular recognition to bind to saccharides for fluorescent detection or selective transport of saccharides across membranes.

Prior art teaches a method for treating fungal infections comprising administering to an animal or plant afflicted with a fungal infection an effective amount of phenylboronic acid or a water soluble derivative thereof. It is known that phenyl boronic acid and derivatives thereof have inhibitory activity toward proteases and lactamases, and it has been reported that it is a substrate for cytochrome p450. It is hypothesized that the substrate effect for cytochrome p450, in combination with its water solubility properties, permits phenyl boronic acid or derivatives thereof to enter a cell and to be degraded by cytochrome p450 to a toxic phenyl compound, which then kills the cell.

In the past 60 years, antibiotics have been critical in the fight against infectious disease caused by bacteria. However, disease-causing bacteria that have become resistant to antibiotic drug therapy are an increasing public health problem. For example, β-lactams belong to a family of antibiotics which is characterized by a β-lactam ring; and bacterial resistance to β-lactams is primarily due to the hydrolysis of the antibiotic by a β-lactamase. Staphylococcus aureus is one of the major resistant pathogens—half of all S. aureus infections in the US are resistant to penicillin, tetracycline and erythromycin. Hence, the development of new families of antibacterial drugs is desirable.

Aromatic boronic acids have been found to be reversible inhibitors of class C β-lactamases and although phenyl boronic acid and derivatives (“PBA's) have been used in combination with certain antibiotics to try and improve their antibiotic effect by inhibiting lactamase enzymes, the prior art does not reveal a direct and significant bactericidal or bacteriostatic effect of PBA's when used alone. For example, minimum inhibitory concentration (“MIC”) levels quoted in prior art in relation to certain aromatic boronic acids that are outside the scope of this specification are typically of the order of about ≧128 μg/mL, whilst in contrast an acceptable MIC for a classical antibiotic like penicillin is at least one order of magnitude lower. Thus, it follows that the prior art actually teaches away from the use of the conjugated boronic acid compounds discussed herein (hereinafter “CBA's”) alone as effective antibiotic agents. CBA's are discussed in detail below. It is, thus, an object to explore conjugated boronic acid compounds and their uses as antibacterial drugs.

### SUMMARY

In one aspect, a pharmaceutical composition for the treatment of a disorder associated with a bacterial infection is provided. The pharmaceutical composition includes a conjugated boronic acid or a derivative thereof; and, a suitable pharmaceutical vehicle.

In some embodiments, the conjugated boronic acid includes a compound in which a boronic acid group is covalently linked with one or more of the following moieties:
a. a carbon atom which is further linked to an additional atom via a double bond, wherein the additional atom is selected from
   i. A carbon atom
   ii. A heteroatom
b. an aromatic ring
c. a polycyclic aromatic structure, or
d. a heterocyclic aromatic structure.

In another aspect, methods of treating disorders associated with bacterial infections are also disclosed. Such methods include administering the pharmaceutical compositions described herein.

In a further aspect a pharmaceutical composition is disclosed. The composition includes:
a. a conjugated boronic acid or a derivative thereof; and
b. a pharmaceutical vehicle including one or more of:
   i. an oil in water emulsion
   ii. an oil in water emulsion, including between 2% and 50% hydrophobic components
   iii. an oil in water emulsion, including between 2% and 50% hydrophobic components, wherein the hydrophobic component is composed of at least two oils
   iv. an oil in water emulsion, including between 2% and 50% hydrophobic components, wherein the hydrophobic component contains silicone
   v. an oil in water emulsion, including between 2% and 50% hydrophobic components, wherein the oil component is composed of at least two oils, wherein the hydrophobic component further contains silicone
   vi. an oil in water emulsion, wherein the surfactant used to stabilize the emulsion is a non-ionic surfactant
   vii. an oil in water emulsion, wherein the surfactant used to stabilize the emulsion is a non-ionic surfactant having an HLB value of more than 9
   viii. an oil in water emulsion, concurrently containing a non-ionic surfactant and a polymeric agent
   ix. an oil in water emulsion, concurrently containing (i) a non-ionic surfactant, (ii) a polymeric agent; and (iii) an agent selected from a fatty alcohol and a fatty acid
   x. a lacquer, suitable for application onto a keratinous surface
   xi. a lacquer, simultaneously containing (i) a volatile solvent; and (ii) a polymeric agent
   xii. a lacquer, simultaneously containing (i) a volatile solvent; and (ii) a film-forming polymeric agent
   xiii. a water in oil emulsion
   xiv. a water in oil emulsion, comprising between 20% and 80% hydrophobic components
   xv. a water in oil emulsion, comprising between 20% and 80% hydrophobic components, wherein the hydrophobic component is composed of at least two oils
   xvi. a water in oil emulsion, comprising between 20% and 80% hydrophobic components, wherein the hydrophobic component contains silicone
   xvii. a water in oil emulsion, comprising between 20% and 80% hydrophobic components, wherein the oil component is composed of at least two oils, wherein the hydrophobic component further contains silicone
   xviii. a water in oil emulsion, wherein the surfactant used to stabilize the emulsion is a non-ionic surfactant
   xix. a water in oil emulsion, wherein the surfactant used to stabilize the emulsion is a non-ionic surfactant having an HLB value of more than 9
   xx. a water in oil emulsion, concurrently containing a non-ionic surfactant and a polymeric agent
   xxi. a water in oil emulsion, concurrently containing (i) a non-ionic surfactant, (ii) a polymeric agent; and (iii) an agent selected from a fatty alcohol and a fatty acid
   xxii. a carrier, comprising at least 60% of a hydrophilic organic solvent
   xxiii. a carrier, comprising at least 60% of a hydrophilic organic solvent, wherein the hydrophilic solvent is not volatile
   xxiv. a carrier, comprising at least 60% of a hydrophilic organic solvent, wherein the hydrophilic solvent is not a lower alcohol
   xxv. a carrier, comprising at least 60% of a hydrophilic organic solvent, wherein the hydrophilic organic solvent is selected from the group consisting of propylene glycol, polyethylene glycol and glycerin
   xxvi. a carrier, comprising at least 60% of a hydrophilic organic solvent, wherein the hydrophilic organic solvent simultaneously contains at least two solvents, selected from the group consisting of propylene glycol, polyethylene glycol and glycerin
   xxvii. a carrier, comprising at least 60% of a hydrophilic organic solvent, wherein the hydrophilic organic solvent simultaneously contains (i) at least two solvents, selected from the group consisting of propylene glycol, polyethylene glycol and glycerin, and (ii) a surfactant
   xxviii. a carrier, comprising at least 60% of a hydrophilic organic solvent, wherein the hydrophilic organic solvent simultaneously contains (i) at least one solvent, selected from the group consisting of propylene glycol, polyethylene glycol and glycerin, (ii) a surfactant, and (iii) a polymeric agent
   xxix. a carrier, comprising at least 60% of a hydrophilic organic solvent, wherein the hydrophilic organic solvent simultaneously contains (i) at least one hydrophilic solvent, selected from the group consisting of propylene glycol, polyethylene glycol and glycerin, (ii) a surfactant, (iii) a polymeric agent and (iv) an agent selected from a fatty alcohol and a fatty acid.
   xxx. a carrier, comprising a hydrophilic organic solvent, wherein the carrier is substantially water-free
   xxxi. a carrier, comprising at least 50% of petrolatum
   xxxii. a carrier, comprising at least 50% of petrolatum, wherein the carrier is substantially water-free
   xxxiii. a carrier, comprising at least 70% of petrolatum
   xxxiv. a carrier, comprising at least 80% of petrolatum
   xxxv. a carrier, simultaneously containing (i) at least 50% of petrolatum, and (ii) a surfactant
   xxxvi. a carrier, simultaneously containing (i) at least 50% of petrolatum, and (ii) a surfactant, wherein the carrier is substantially water-free
   xxxvii. a carrier, simultaneously containing (i) at least 50% of petrolatum, (ii) a surfactant and (iii) a polymeric agent and
   xxxviii. a carrier, simultaneously containing (i) at least 50% of petrolatum, (ii) a surfactant, (iii) a polymeric agent and (iv) an agent selected from a fatty alcohol and a fatty acid.

In a still further aspect a pharmaceutical composition is disclosed. The composition includes a conjugated boronic acid or a derivative thereof and a pharmaceutical vehicle suitable for treatment of a disorder associated with a bacterial infection.

In some embodiments, the conjugated boronic acid is an unsubstituted or substituted phenyl group, having the general formula

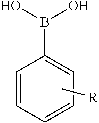

wherein the aromatic ring is linked to one OH group or to R, wherein R comprises H, alkyl, alkenyl, benzyl, CHO, OR′, NHR′, halogen, CONHR′ or COOR′; and R′ comprises H, alkyl, aryl, alkenyl, or benzyl.

### DESCRIPTION

This application relates to CBA's and derivatives thereof and their uses as effective antibacterial agents in their own right in the absence of other antibiotics. The application further relates to different non limiting emollient vehicles as carriers for the CBA's; and also discusses the effect of pH on the CBA's.

It has been surprisingly discovered that conjugated boronic acid compounds, and derivatives thereof are not only effective against microbiological species, other than fungi, yeasts or molds but they are also able to act against bacterial pathogens with known resistance. In particular the striking effect of CBV solutions on different bacterial microorganisms is discussed.

The description further offers a method of therapy of a disorder, associated with an infection by a microbiological species other than fungi, yeasts or molds, by administering to a mammal in nead a therapeutically effective dose of a CBA.

In the context herein, “therapy” or “treatment”, are terms that can be applied to any form of treatment for any illness or disorder, including but not limited to curing an illness or disorder; improving the condition of an illness or disorder; reducing the symptoms of an illness or disorder; preventing the complications of an illness or disorder; and directly or indirectly preventing an illness or disorder.

### Conjugated Boronic Acid (CBA)

A CBA is a compound, wherein a boronic acid group is covalently linked to a carbon atom, wherein such carbon atom is further linked to an additional atom via a double bond.

In an embodiment herein, the CBA comprises a boronic acid group, covalently linked to an aromatic ring.

In an embodiment herein, the CBA is a phenyl boronic acid (Hereinafter “PBA”), having the general formula

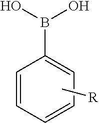

wherein the aromatic ring is linked to one OH group, or to at least one R group, selected from the group consisting of H, alkyl, alkenyl, benzyl, CHO, OR′, NHR′, a halogen, F, Cl, Br, CONHR′ and COOR′; R″ is selected from the group consisting of H, alkyl, aryl, alkenyl, and benzyl.

“Halogen” as used herein includes, without limitation, F, Cl or Br.

As used herein, “alkyl” includes, without limitation, C₁-C₁₀ alkyl, C₁-C₁₀ alkyl, C₁-C₈ alkyl, C₁-C₆ alkyl, C₁-C₄ alkyl, and C₁-C₃ alkyl. In some embodiments, “alkyl” includes, without limitation, branched or unbranched methyl, ethyl, propyl, butyl, pentyl, and hexyl.

As used herein, “alkenyl” includes, without limitation, C₂-C₁₀ alkenyl, C₂-C₁₀ alkenyl, C₂-C₈ alkenyl, C₂-C₆ alkenyl, C₂-C₄ alkyenl, and C₂-C₃ alkenyl. In some embodiments, “alkenyl” includes, without limitation, branched and unbranched ethenyl, butenyl, pentenyl, and hexenyl.

The term “aryl” as used herein refers to an aromatic species containing 1 to 3 aromatic rings, either fused or linked. In one embodiment, the aryl group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (Cᵣ C₆)-alkyl; and wherein each V is independently a bond or (C₁-C₆)-alkyl.

In specific embodiments, the PBA is a formyl phenyl boronic acid or a derivative thereof. In certain embodiments, the formyl phenyl boronic acid is a 2-formyl phenyl boronic acid or a derivative thereof.

In certain embodiments, the CBA comprises a boronic acid group, covalently linked to a polycyclic aromatic structure.

In certain embodiments, the CBA comprises a boronic acid group, covalently linked to a heterocyclic aromatic structure, wherein the heteroatom is O or N, as exemplified by the following compounds:

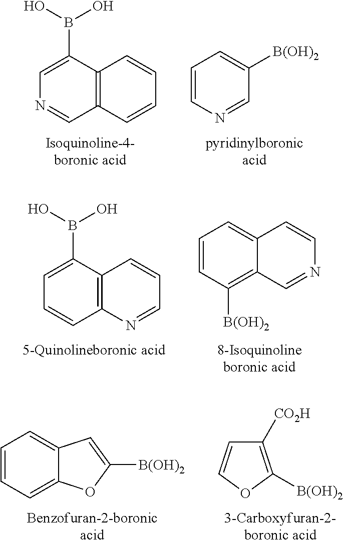

### Microbial Species Suitable for Treatment by a CBA

In accordance with the description herein, the CBA is effective against bacteria. Bacteria are distinct from fungi in multiple ways: Fungi are eukaryotes and bacteria are prokaryotes. Bacteria are single celled; whereas most fungi are multicellular but some fungi are single celled (like yeast). Both possess cell walls but the components within the cell walls are different (fungi have chitin in their cell wall, the same component found in the exoskeleton of insects). Various antifungal agents work by inhibition of the fungal cytochrome P450 14α-demethylase, thereby interrupting the conversion of lanosterol to ergosterol, a component of the fungal cell membrane; however, the same mechanism cannot be employed for killing or inhibiting bacteria.

Bacteria are microorganisms that have circular double-stranded DNA and (except for Mycoplasma sp) cell walls. Bacteria are classified by several criteria, including morphology. They may be cylindric (bacilli), spherical (cocci), or spiral (spirochetes). Gram-positive bacteria retain crystal violet dye after iodine fixation and alcohol decolorization, whereas gram-negative bacteria do not. Gram-negative bacteria have an additional outer membrane containing lipopolysaccharide (endotoxin).

The following Table A provides a classification of common pathogenic bacteria

TABLE A

| Aerobic vs | | |
|---|---|---|
| Anaerobic | Type | Organism |
| Aerobic | Gram-positive cocci, | Staphylococcus aureus (coagulase- |
| | catalase-positive | positive), S. epidermidis |
| | | (coagulase-negative), other |
| | | coagulase-negative staphylococci |
| Aerobic | Gram-positive cocci, | Enterococcus faecalis, E. faecium, |
| | catalase-negative | Streptococcus agalactiae (Group B |
| | | streptococcus), S. bovis, |
| | | S. pneumoniae, S. pyogenes |
| | | (Group A streptococcus), Viridans |
| | | group streptococci, S. anginosus, |
| | | S. mutans |
| Aerobic | Gram-negative cocci | Moraxella catarrhalis, Neisseria |
| | | gonorrhoeae, N. meningitidis |
| Aerobic | Gram-positive bacilli | Bacillus anthracis, Corynebacterium |
| | | diphtheriae, C. jeikeium, |
| | | Erysipelothrix rhusiopathiae, |
| | | Gardnerella vaginalis |
| | | (gram-variable) |
| Aerobic | Acid-fast bacilli | Mycobacterium avium complex, |
| | | Mycobacterium kansasii, M. leprae, |
| | | M. tuberculosis, Nocardia sp |
| Aerobic | Gram-negative bacilli | Enterobacteriaceae (Citrobacter sp, |
| | | Enterobacter aerogenes, |
| | | Escherichia coli, Klebsiella sp, |
| | | Morganella morganii, Proteus sp, |
| | | Providencia rettgeri, Salmonella |
| | | typhi, other Salmonella sp, Serratia |
| | | marcescens, Shigella sp, Yersinia |
| | | enterocolitica, Y. pestis) |
| Aerobic | Fermentative, non- | Aeromonas hydrophila, |
| | Enterobacteriaceae | Chromobacterium violaceum, |
| | | Plesiomonas shigelloides, Pasturella |
| | | multocida, Vibrio cholerae, |
| | | V. vulnificus |
| Aerobic | Non-fermentative, | Acinetobacter calcoaceticus, |
| | non- | Flavobacterium meningosepticum, |
| | Enterobacteriaceae | Pseudomonas aeruginosa, |
| | | Pseudomonas alcaligenes, other |
| | | Pseudomonas sp, Stenotrophomonas |
| | | maltophilia |
| Aerobic | Fastidious gram- | Actinobacillus |
| | negative coccobacilli | actinomycetemcomitans, Bartonella |
| | and bacilli | bacilliformis, B. henselae, |
| | | B. quintana, Brucella sp, Bordetella |
| | | sp, Eikenella corrodens, |
| | | Haemophilus influenzae, other |
| | | Haemophilus sp, Legionella sp |
| Aerobic | Curved bacilli | Campylobacter jejuni, Helicobacter |
| | | pylori |
| Aerobic | Chlamydiaceae | Chlamydia trachomatis, |
| | | Chlamydophila pneumoniae, |
| | | C. psittaci |
| Aerobic | Rickettsiae | Rickettsia prowazekii, R. rickettsii |
| Aerobic | Mycoplasma | Mycoplasma pneumoniae |
| Aerobic | Treponemataceae | Borrelia burgdorferi, Leptospira sp, |
| | (spiral organisms) | Treponema pallidum |
| Anaerobic | Gram-negative bacilli | Bacteroides fragilis, other |
| | | Bacteroides sp, Fusobacterium sp, |
| | | Prevotella sp |
| Anaerobic | Gram-negative cocci | Veillonella sp |
| Anaerobic | Non-spore-forming | Actinomyces sp, Bifidobacterium sp, |
| | gram-positive bacilli | Eubacterium sp, Propionibacterium |
| | | sp |
| Anaerobic | Endospore-forming | Clostridium botulinum, |
| | gram-positive bacilli | C. perfringens, C. tetani, other |
| | | Clostridium sp |
| Anaerobic | Gram-positive cocci | Gemella morbillorum, Peptococcus |
| | | niger, Peptostreptococcus sp |

Propionibacterium acnes is a non-sporulating bacilliform (rod-shaped), gram-positive bacterium found in a variety of locations on the human body including the skin, mouth, urinary tract and areas of the large intestine. P. acnes is most commonly associated with its implicated role as the predominant cause of the common inflammatory skin condition Acne vulgaris. It has also been found in corneal ulcers, infections of joints and suture sites. Tetracycline-resistant P. acnes is now quite common. It is primarily anaerobic. The role of P. acnes in human pathology is complex.

### Fields of Applications

Based on our in-vitro studies, we may conclude that CBAs are effective against Gram positive bacteria, (Staphilococus aurus) and Gram negative bacteria (Escherichia coli and Pseudomonas aeruginosa). Additionally, it has been shown that CBAs kill the anaerobic bacteria, Propionbacterium acnes.

Based on their broad spectrum of activity, CDAs can be used for the treatment of a variety of disorders that involve bacterial infection as an etiological factor. Examples of conditions, suitable for treatment using a CDA include:

1. Conditions Associated with Anaerobic Bacteria

| Condition | Bacteria |
|---|---|
| Soft-tissue infection: crepitant cellulitis, myositis, | C. perfringens |
| clostridial myonecrosis, hemolysis, muscle necrosis, | |
| food poisoning, enteritis necroticans | |
| Antibiotic-associated colitis | C. difficile |
| Neutropenic enterocolitis, Colorectal malignancy, | C. septicum |
| Hemolysis by septicolysine, Tissue necrosis, DNA | |
| lysis by DNase, Hyaluronan lysis by hyaluronilase | |
| Tetanus | C. tetani |
| Botulism | C. botulinum |
| Abdominal infections: Cholecystitis, peritonitis, | C. perfringens, |
| ruptured appendix, bowel perforation, neutropenic | C. ramosum |
| enterocolitis | |

Anaerobic bacteria are intolerant of O₂, replicating at low oxidation-reduction potential sites, such as necrotic, devascularized tissue. In humans, anaerobic organisms are among the normal flora (especially of the GI tract, mouth, and vagina), but when they enter sterile spaces, they can cause serious infections.

The most notorious of the anaerobic pathogens are the clostridia—spore-forming, gram-positive bacilli. The pathogenic species produce tissue-destructive and neural exotoxins that are responsible for disease manifestations.

Other anaerobes of concern include actinomyces israelii, a cause of chronic localized or hematogenous infection, and a host of nonsporulating anaerobes, both cocci and bacilli, most of which are commensals until they invade normally sterile spaces.

2. Conditions Associated with Gram-Positive Bacilli

Gram-positive bacilli cause anthrax, diphtheria, erysipelothricosis, listeriosis, and nocardiosis. Serious symptoms from anthrax and diphtheria are due to powerful toxins produced by the organisms.

3. Conditions Associated with Gram-Positive Cocci

Many gram-positive cocci are commensal organisms that cause infection only when they find their way into normally sterile areas. They are the most common cause of skin infections and a frequent cause of pneumonia and septicemia. Although generally susceptible to a broad range of antibiotics, certain strains have developed resistance to every available antimicrobial agent.

Streptococcus pneumoniae (pneumococcus) is a gram-positive, aerobic organisms. In the US, pneumococcal infection annually causes about 7 million cases of otitis media, 500,000 cases of pneumonia, 50,000 cases of sepsis, 3,000 cases of meningitis, and 40,000 deaths. Staphylococcus aureus is the most pathogenic; it typically causes skin infections and sometimes pneumonia, endocarditis, and osteomyelitis. Some strains elaborate toxins that cause gastroenteritis, scalded skin syndrome, and toxic shock syndrome. Staphylococci cause disease by direct tissue invasion and sometimes by exotoxin production. S. aureus bacteremia, which frequently causes metastatic foci of infection, may occur with any localized staphylococcal infection but is particularly common with infection related to intravascular catheters or other foreign bodies. Skin infections are the most common form of staphylococcal disease. Superficial infections may be diffuse, with vesicular pustules and crusting (impetigo) or sometimes cellulitis, or focal and nodular (furuncles and carbuncles). Deeper cutaneous abscesses are common. Staphylococci are commonly implicated in wound and burn infections, postoperative incision infections, and mastitis or breast abscess in nursing mothers. Neonatal infections usually appear within 6 wk after birth and include skin lesions with or without exfoliation, bacteremia, meningitis, and pneumonia. S. aureus is a common cause of hospital-acquired pneumonia. Staphylococcal pneumonia is occasionally characterized by formation of lung abscesses followed by rapid development of pneumatoceles and empyema.

Endocarditis develops, particularly in IV drug abusers and patients with prosthetic heart valves. It is an acute febrile illness often accompanied by abscesses, embolic phenomena, pericarditis, subungual petechiae, subconjunctival hemorrhage, purpuric lesions, heart murmurs, and valvular heart failure.

Osteomyelitis occurs more commonly in children, causing chills, fever, and pain over the involved bone. Redness and swelling subsequently appear. Periarticular infection frequently results in effusion, suggesting septic arthritis rather than osteomyelitis.

Staphylococci may produce multiple toxins, which trigger cytokine release from certain T cells, causing serious systemic effects, including skin lesions, shock, organ failure, and death. Toxic shock syndrome may occur from use of vaginal tampons or as a complication of a seemingly minor postoperative infection.

Staphylococcal scalded skin syndrome, which is caused by several toxins termed exfoliatins, is an exfoliative dermatitis of childhood characterized by large bullae and peeling of the upper layer of the skin. Eventually, exfoliation occurs.

Staphylococcal food poisoning is caused by ingesting a preformed heat-stable staphylococcal enterotoxin.

Streptococcal and Enterococcal Infections: Streptococci are gram-positive aerobic organisms that cause many disorders, including pharyngitis, pneumonia, wound and skin infections, sepsis, and endocarditis. Symptoms vary with the organ infected. Many streptococci elaborate virulence factors, including streptolysins, DNases, and hyaluronidase, which contribute to tissue destruction and spread of infection. A few strains release exotoxins that activate certain T cells, triggering release of cytokines, including tumor necrosis factor-α, interleukins, and other immunomodulators, which activate the complement, coagulation, and fibrinolytic systems, in turn leading to shock, organ failure, and death.

Common streptococcal diseases include pharyngitis and skin infections. Skin infections include impetigo and cellulites. Necrotizing fasciitis due to S. pyogenes is a severe dermal (or rarely muscular) infection that spreads along fascial planes.

Septicemia, puerperal sepsis, endocarditis, and pneumonias due to streptococci remain serious complications, especially if the organism is a multiresistant enterococcus.

Enterococcus faecalis and E. faecium cause endocarditis, UTI, intra-abdominal infection, cellulitis, and wound infection as well as concurrent bacteremia.

4. Conditions Associated with Gram-Negative Bacilli

Gram-negative bacilli are responsible for numerous diseases. Some are commensal organisms found among normal intestinal flora. These commensal organisms plus others, from animal or environmental reservoirs, may cause disease. UTIs, diarrhea, peritonitis, and bloodstream infections are commonly caused by gram-negative bacilli. Plague, cholera, and typhoid fever are rare but serious gram-negative infections, as exemplified below:

Escherichia coli are the most numerous aerobic commensal inhabitants of the large intestine. Certain strains produce toxins that cause diarrhea, and all strains produce infection when they invade sterile tissues.

Klebsiella, Enterobacter, and Serratia are closely related normal intestinal flora that rarely cause disease in normal hosts. Infections with Klebsiella, Enterobacter, and Serratia are usually hospital-acquired and occur mainly in patients with diminished resistance. Usually, Klebsiella, Enterobacter, and Serratia cause infections in the respiratory or urinary tract that present as pneumonia, cystitis, or pyelitis and may progress to lung abscess, empyema, and septicemia. Serratia, particularly S. marcescens, has greater affinity for the urinary tract. Enterobacter can cause otitis media, cellulitis, and neonatal sepsis.

Pseudomonas aeruginosa and other members of this group of gram-negative bacilli are opportunistic pathogens that frequently cause hospital-acquired infections, particularly in ventilator patients, burn patients, and those with chronic debility. Many sites can be infected, and infection is usually severe.

Typhoid fever is a systemic disease caused by Salmonella typhi. Shigellosis is an acute infection of the intestine caused by Shigella sp.

5. Conditions Associated with Micobacteria

Mycobacteria are small, slow-growing, aerobic bacilli distinguished by a complex, lipid-rich cell envelope responsible for their characterization as “acid-fast” (i.e., resistant to decolorization by acid after staining with carbolfuchsin). The most common mycobacterial infection is tuberculosis; others include leprosy and various diseases caused by Mycobacterium avium complex.

### 6. Bacterial Skin Infections

Bacterial skin infections treatable with the compositions described herein include, without limitation:
Acne, involving Propionibacterium acnes
Rosacea, responsive to antibiotics
Cellulitis, an acute bacterial infection of the skin and subcutaneous tissue most often caused by streptococci and staphylococci.
Cutaneous abscess, a localized collection of pus in the skin, wherein the most common organisms are Staphylococcus aureus and streptococci.
Erysipelas is a type of superficial cellulitis (see Bacterial Skin Infections: Cellulitis) with dermal lymphatic involvement.
Erysipelas, characterized clinically by shiny, raised, indurated, and tender plaque-like lesions with distinct margins, most often caused by streptococci but can also involve staphylococcus aureus, klebsiella pneumoniae, haemophilus influenzae, escherichia coli, S. warneri, streptococcus pneumoniae, S. pyogenes, and moraxella sp.
Folliculitis, which is usually caused by staphylococcus aureus but occasionally pseudomonas aeruginosa.
Furuncles
Impetigo and eethyma, superficial skin infection with crusting or bullae caused by streptococci, staphylococci.
Necrotizing subcutaneous infection, typically caused by a mixture of aerobic and anaerobic organisms that cause necrosis of subcutaneous tissue.

### 7. Sexually Transmitted Bacterial Infections

Bacterial sexually transmitted diseases (STDs) include syphilis, gonorrhea, chancroid, lymphogranuloma venereum, granuloma inguinale, and syndromes caused by chlamydia, mycoplasma, and ureaplasma infections. Trichomoniasis is infection of the vagina or male genital tract with Trichomonas vaginalis. Bacterial vaginosis, the most prevalent cause of vaginal discharge or malodor, is a polymicrobial clinical syndrome resulting from replacement of the normal Lactobacillus sp. in the vagina with high concentrations of anaerobic bacteria (e.g., Prevotella sp. and Mobiluncus sp.), G. vaginalis, and Mycoplasma hominis.

### 8. Ear Infection

External otitis is usually caused by bacteria, such as Pseudomonas aeruginosa, Proteus vulgaris, Staphylococcus aureus, or Escherichia coli. Otitis media may involve Escherichia coli, and Staphylococcus aureus, Streptococcus pneumoniae, Moraxella (Branhamella) catarrhalis and Haemophilus influenzae.

### Route of Administration

The CBA and derivatives thereof are suitable for administration directly or indirectly to an inflicted area, in need of treatment, through the following routes of administration:

1. Topical administration: for local effect, the CBA is applied directly where its action is desired;
2. Enteral: when the desired effect is systemic (non-local), the CBA is given via the digestive tract; and
3. Parenteral: when the desired effect is systemic, the CBA is given by other routes than the digestive tract The following list more specifically exemplifies some routes of administration.

### 1. Topical

Topical administration is any form of administration that reaches a body organ topically, such as epicutaneous administration (application onto the skin), inhalation, enema, eye drops (onto the conjunctiva), ear drops, intranasal (into the nose) and vaginal.

Exemplary dosage forms that are suitable for topical administration of CBA include cream, gel, liniment, lotion, ointment, paste, spray, foam, mousse, lacquer (e.g., for nail treatment) and transdermal patch. Additionally, topical vaginal dosage forms may include a douche, an intrauterine device, a pessary (vaginal suppository), a vaginal ring and a vaginal tablet. Rectal dosage forms include enema and suppositories. Inhaled dosage forms include aerosol inhalers, metered dose inhalers and solutions for nebulizer. Ophthalmic dosage forms include eye drop (solution or suspension), ophthalmic gel and ophthalmic ointment. In a preferred embodiment the dosage form is a foam that is thermally stable and breakable under sheer force but is not “quick breaking which allows comfortable application and well directed administration to the target area.

### 2. Enteral

Enteral is any form of administration that involves any part of the gastrointestinal tract by mouth (orally), as buccal or sublingual tablets, capsules, suspensions, solutions, powder or drops; by gastric feeding tube, duodenal feeding tube, or gastrostomy; and rectally, in suppository or enema form.

### 3. Parenteral by Injection or Infusion

Intravenous (into a vein); intraarterial (into an artery); intramuscular (into a muscle); intracardiac (into the heart); subcutaneous (under the skin); intraosseous infusion (into the bone marrow); intradermal, (into the skin itself); intrathecal (into the spinal canal); and intraperitoneal.

### 4. Other Parenteral

Transdermal (diffusion through the intact skin); transmucosal (diffusion through a mucous membrane), e.g. insufflation (snorting), sublingual, buccal (absorbed through cheek near gumline) and vaginal; and inhalational; epidural (synonym: peridural) (injection or infusion into the epidural space); and intravitreal.

### Compositions for Topical Administration Comprising a CBA-Foams

A general procedure for preparing foamable compositions is set out in WO 2004/037225, which is incorporated herein by reference. Foamable compositions and methods are also described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 0/815948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, as well as U.S. Pat. No. 6,730,288, entitled MOUSSE COMPOSITION, all of which are incorporated by reference in their entirety herein. More particularly any of the active ingredients; the solvents; the surfactants; foam adjuvants; polymeric agents, penetration enhancers; preservatives, humectants; moisturizers; and other excipients as well as the propellants listed therein can be applied herein and are incorporated by reference.

### EXAMPLES

This invention is not limited to these examples and experiments. Many variations will suggest themselves and are within the full intended scope of the appended claims.

### Example 1

### Antimicrobial Efficacy Testing of 2-Formylphenyl Boronic Acid

The purpose of this study was to assess the antimicrobial efficacy of 2-Formylphenyl boronic acid (FPB) aqueous solutions, in different concentrations of 10 mM, 20 mM and 40 mM. 2-FBP was obtained from Sigma Aldrich.

Test Procedure: The test consisted of challenging the test solutions with specified microorganisms, storing the inoculated preparations at a prescribed temperature, removing the inoculated samples at specified intervals of time and counting the number of viable organisms in the withdrawn samples using a plate count procedure. The technical details were as follows:
The test was conducted in 20 g samples.
Each sample was separately inoculated by one of the test organisms.
The inoculated containers were incubated at 25° C. together with an uninoculated sample.
The inoculated container with P. acnes was incubated under anaerobic conditions at the same temperature.
The culture media for P. acnes was Brain Heart Infusion Agar instead of Tryptic Soy Agar.

Formulations were challenged by introducing the following microorganisms:

Escherichia coli (ATCC No. 8739)

Staphylococcus aureus (ATCC No. 6538)

Pseudomonas aeruginosa (ATCC No. 9027)

Propionbacterium acnes ATCC 11827

Counting of microorganisms in the inoculated samples was performed 1, 2 and 7 days following inoculation. The number of colony-forming units (cfu/g) determined at each incubation time point was compared to the number of cfu/g measured in non-inoculated control samples of FPB solutions.

Antimicrobial effectiveness results, expressed as colony forming units of surviving microorganisms in the FPB solutions (cfu/g), are detailed in Tables 1-3.

Solution 1 (10 mM FPB) completely destroyed P. acnes following one day of exposure. It was also effective against E. coli, Staph. aureus and P. aeruginosa, with 2.0 log reduction of the count from the initial lad within two days of exposure (Table 1). Solution 2 (20 mM FBA) and Solution 3 (40 mM FBA) completely eradicated all microbial strains (E. coli, Staph. Aureus, P. Aeruginosa and P. acnes following one day of exposure (Table 2-3). In conclusion, 2-Formylphenyl boronic acid solutions, in concentrations of 10 mM, 20 mM and 40 mM, was effective against E. coli, Staph. aureus, P. aeruginosa and P. acnes.

TABLE 1
Antimicrobial Effectiveness of 10 mM FPB

| | | Initial | No. of Surviving | | |
|---|---|---|---|---|---|
| | | Contamination | Microorganisms CFU/g | | |
| Test Organisms | | CFU/g | 1 days | 2 days | 1 week |
| E. coli | 8739 | 2.8 × 10⁵ | 6.4 × 10⁴ | <10 | <10 |
| Staph. aureus | 6538 | 5.2 × 10⁵ | 4.5 × 10⁴ | 5.4 × 10² | <10 |
| Ps. aeruginosa | 9027 | 4.3 × 10⁶ | 4.3 × 10⁴ | 5.0 × 10¹ | <10 |
| P. acnea | | 4.1 × 10⁵ | <10 | <10 | <10 |

| | | | | |
|---|---|---|---|---|
| Uninoculated Control | — | <10 | <10 | <10 |

TABLE 2
Antimicrobial Effectiveness of 20 mM FPB

| | | Initial | No. of Surviving | | |
|---|---|---|---|---|---|
| | | Contamination | Microorganisms CFU/g | | |
| Test Organisms | | CFU/g | 1 days | 2 days | 1 week |
| E. coli | 8739 | 2.8 × 10⁵ | 7.6 × 10³ | <10 | <10 |
| Staph. aureus | 6538 | 5.2 × 10⁵ | <10 | <10 | <10 |
| Ps. aeruginosa | 9027 | 4.3 × 10⁶ | <10 | <10 | <10 |
| P. acnes | | 4.1 × 10⁵ | <10 | <10 | <10 |

| | | | | |
|---|---|---|---|---|
| Uninoculated Control | — | <10 | <10 | <10 |

TABLE 3
Antimicrobial Effectiveness of 30 mM FPB

| | | Initial | No. of Surviving | | |
|---|---|---|---|---|---|
| | | Contamination | Microorganisms CFU/g | | |
| Test Organisms | | CFU/g | 1 days | 2 days | 1 week |
| E. coli | 8739 | 2.8 × 10⁵ | <10 | <10 | <10 |
| Staph. aureus | 6538 | 5.2 × 10⁵ | <10 | <10 | <10 |
| Ps. aeruginosa | 9027 | 4.3 × 10⁶ | <10 | <10 | <10 |
| P. acnes | | 4.1 × 10⁵ | <10 | <10 | <10 |

| | | | | |
|---|---|---|---|---|
| Uninoculated Control | — | <10 | <10 | <10 |

### Example 2

### A Foamable Oil in Water Emulsion Pharmaceutical Composition Comprising 2-Formyl Phenyl Boronic Acid at 20 Mm and 40 Mm, which can be Used to Make a Foam and which can Also be Used as a Lotion, if Formulated without the Addition of Propellant

### Emulsion Carrier

| Ingredients | % w/w |
|---|---|
| light mineral oil | 6.00 |
| Isopropyl myristate | 6.00 |
| Glycerol monostearate | 0.50 |
| PEG-40 stearate | 3.00 |
| Stearyl alcohol | 1.00 |
| Xanthan gum | 0.30 |
| Methocel K100M | 0.30 |
| Polysorbate 80 | 1.00 |
| Purified water | 81.30 |
| Sharomix 824 | 0.60 |
| Total: | 100.00 |

### Manufacturing Procedure:

### Oily Phase:

1. Mix light mineral oil, Isopropyl myristate, Glycerol monostearate and Stearyl alcohol and heat to 65-70 C. to complete dissolution (clear solution).

### Water Phase:

1. Cool about ¼ of water to ˜5C.
2. Heat rest of water to ˜90 C. and add Methocel K100M while vigorously mixing for dispersion (about 20 min.)
3. Pour cold water to hot water while mixing (about 20 min.)
4. Heat water to about 60 C. and add Xanthan gum while mixing (about 20 min.)
5. At 60-65 C. add PEG-40 stearate and Polysorbate 80 while mixing (about 10 min.)

### Emulsification:

1. At 60-65 C., Add the water phase to the oily phase while mixing (about 10 min.).

### 2. Cool to 40 C. and add Sharomix 824.

3. Cool to RT and add water, if necessary.

### Lotion Composition

| | |
|---|---|
| Emulsion Carrier | 15 grams |
| 2-Formylphenyl boronic acid | 45 MG (20 mM) |

### Properties:

| | |
|---|---|
| Microscope | No Crystals |
| Color | White |
| Odor | Very Faint Odor |

### Foam Composition

| | |
|---|---|
| Emulsion Carrier | 30 grams |
| 2-Formylphenyl boronic acid | 180 mg (40 mM) |
| Propellant (AP-70) propane, butane, isobutane | 2.40 grams |

### Properties:

| | |
|---|---|
| Foam quality | Excellent |
| Color | White |
| Odor | Very faint odor |
| Microscope | No crystals |
| Shakability of formulation including propellant | Good |

### Comments:

The active agent FPB is soluble in the carrier.

FPB and does not interfere with the formation of an emulsion, which can be used as a lotion.

FPB and does not interfere with foam formation.

By “excellent” foam quality is meant a foam that is very rich and creamy in appearance, does not show any bubble structure or shows a very fine (small) bubble structure; does not rapidly become dull; and upon spreading on the skin, the foam retains the creaminess property and does not appear watery.

“Shakability” means that the composition contains some or sufficient flow to allow the composition to be mixed or remixed on shaking. That is, it has fluid or semi fluid properties. In some very limited cases it may still be possible to have a foamable composition which is flowable but not apparently shakable.

### Example 3

### A Gel Pharmaceutical Composition Comprising 2-Formylphenyl Boronic Acid at 20 mM and 40 mM, Which can be Used to Make an Excellent Foam and which can Also be Used as a Gel, if Formulated without the Addition of Propellant

### Gel Carrier Composition

| Ingredients | % w/w |
|---|---|
| Cyclomethicone | 1.00 |
| PEG-40 Stearate | 3.00 |
| Polysorbate 60 | 1.00 |
| Glyceryl monostearate | 1.00 |
| Stearyl alcohol | 1.00 |
| Carbomer 981 | 0.22 |
| NaOH (18% aqueous solution) | 0.22 |
| Hydroxypropyl methylcellulose | 0.25 |
| Xanthan gum | 0.25 |
| Citric acid | 0.31 |
| Sodium citrate tribasic dehydrate | 0.44 |
| Purified water | To 100.00 |

### Manufacturing Procedure:

### Oil Phase:

Mix Cyclomethicone, glyceryl monostearate and stearyl alcohol and heat to 65-70° C. to complete dissolution (clear solution).

### Water Phase (A):

1. Cool about ¼ of water to ˜5° C.
2. Heat about ⅓ of water to ˜90C. and add Methocel K100M while vigorously mixing for dispersion (about 20 min.)
3. Pour cold water to hot water while mixing (about 20 min.)
4. Heat water to about 60° C. and add Xanthan gum while mixing (about 20 min.)
5. At 60-65° C. add PEG-40 stearate and Polysorbate 60 while mixing (about 10 min.)

### Water Phase (B):

1. At ˜40C. Add Carbomer 981 to rest of water while vigorously mixing (homogenization). Nix to solubility.
2. Add NaOH 18% solution to neutralization (highly viscous gel accepts). Heat to 60-65° C.

### Emulsification:

1. At 60-65° C., add the two water phases (A+B) to the oil phase while mixing (about 10 min).
2. Cool to 40° C. and add citric acid and sodium citrate tribasic dihydrate.
3. Cool to RT and add water if necessary.

### Gel Composition Comprising FBA

| | |
|---|---|
| Gel carrier composition | 15 grams |
| 2-Formylphenyl boronic acid | 45 MG (20 mM) |

### Properties

| | |
|---|---|
| Microscope | No Crystals |
| Color | White |
| Odor | Very Faint Odor |

### Foam Composition Comprising FBA

| | |
|---|---|
| Gel carrier composition | 30 grams |
| 2-Formylphenyl boronic acid | 180 mg (40 mM) |
| Propellant (AP-70) propane, butane, isobutane | 2.40 grams |

### Properties

| | |
|---|---|
| Foam quality | Excellent |
| Color | White |
| Odor | Very Faint Odor |
| Microscope (crystals only) | No Crystals |
| Shakability of formulation including propellant | Good |

Comments: The results show that the active agent FPB is soluble in the carrier and does not interfere with foam formation and can also be used as a gel.

### Example 4

### Factors that Influence the Stability 2-Formylphenyl Boronic Acid in a Composition

2-Formylphenyl Boronic Acid (2-FBA) was exposed to various formulation conditions, in order to assess its optimized formulation stability properties. The amount of 2-FBA was assessed prior to the experiment and following the exposure to the different conditions by chromatography. The conditions are detailed in the following Table. As shown in the Table, 2-FBA was relatively stable in the presence of NaOH (0.1 N), UV light and heat and it underwent degradation in the presence of HCl (0.1 N) and H₂O₂.

| Sample | Treatment | Duration | Final treatment | % Recovery |
|---|---|---|---|---|
| Untreated | — | — | — | |
| NaOH (0.1N) | 0.5 mL | 30 min | HCl (0.1N) | 96.4 |
| | | | 0.5 mL | |
| HCl (0.1N) | 0.5 mL | 30 min | NaOH (0.1N) | 78.0 |
| | | | 0.5 mL | |
| H₂O₂ | 0.5 mL | 30 min | — | 0.0 |
| UV | Day light | 3 hrs | — | 94.1 |
| Heat | Water bath | 1 hr | Cool to RT | 95.6 |
| | 60° C. | | | |

Hence, in certain embodiments, a pharmaceutical composition comprising 2-FBA comprises:
a pH-modifying system or a buffer system, suitable for maintaining the pH in the range between about 5.5 and about 9; or
a radical scavenger or an antioxidant, which is suitable to protect the; or
a simultaneous combination of (i) a pH-modifying system or a buffer system, suitable for maintaining the pH in the range between about 5.5 and about 9; and (ii) a radical scavenger or an antioxidant.

What is claimed is:

1. A pharmaceutical composition, for the treatment of a disorder associated with a bacterial infection, comprising:
a. a conjugated boronic acid or a derivative thereof; and
b. a suitable pharmaceutical vehicle

2. The composition of claim 1, wherein the conjugated boronic acid comprises a compound wherein a boronic acid group is covalently linked with a moiety selected from the group consisting of:
e. a carbon atom which is further linked to an additional atom via a double bond, wherein the additional atom is selected from
i. A carbon atom
ii. A heteroatom
f. an aromatic ring
g. a polycyclic aromatic structure
h. a heterocyclic aromatic structure.

3. The composition of claim 1, wherein the conjugated boronic acid comprises a formyl phenyl boronic acid.

4. The composition of claim 3, wherein the formyl phenyl boronic acid comprises 2-formyl phenyl boronic acid.

5. The composition of claim 1, wherein the form pharmaceutical composition is selected from the group consisting of:
a. a topical dosage form;
b. an enteral dosage form;
c. a parenteral dosage form;
d. a dosage form, suitable for epicutaneous, inhalation, rectal, ophthalmic, ear, intranasal or vaginal administration;
e. a dosage form comprising cream, gel, liniment, lotion, ointment, paste, spray, foam, mousse, lacquer or a transdermal patch;
f. a dosage form comprising a douche, an intrauterine device, a pessary, a vaginal ring, a vaginal tablet, enema, a rectal suppository, an aerosol inhaler, a metered dose inhaler, a solution for nebulizer, eye drops, an ophthalmic gel or an ophthalmic ointment;
g. a dosage form comprising a buccal tablet, a sublingual tablet, a capsule, a suspension, a solution, a powder or drops;
h. a dosage form, suitable for injection or infusion
i. a dosage form, suitable for parenteral administration through a route selected from the group consisting of intravenous, intraarterial, intramuscular, intracardiac, subcutaneous; intraosseous infusion, intradermal, intrathecal and intraperitoneal.
j. a dosage form, suitable for parenteral administration through a route selected from the group consisting of transdermal, transmucosal, sublingual, buccal, vaginal, inhalational, epidural and intravitreal.
k. a dosage form, containing a pH-modifying system or a buffer system, suitable for maintaining the pH in the range between about 5.5 and about 9.
l. a dosage form, containing a radical scavenger or an antioxidant.
m. a dosage form, simultaneously containing (i) a pH-modifying system or a buffer system, suitable for maintaining the pH in the range between about 5.5 and about 9; and (ii) a radical scavenger or an antioxidant.

6. The composition of claim 1, wherein the disorder is associated with bacteria comprising bacilli, cocci, spirochetes, gram-positive bacteria, gram-negative bacteria, aerobic bacteria or anaerobic bacteria.

7. The composition of claim 1, wherein the disorder is associated with bacteria comprising a staphylococcus, a streptococcus, an enterococcus, Escherichia coli, Klebsiella, Escherichia coli, Enterobacter, Serratia or Pseudomonas aeruginosa.

8. The composition of claim 6, wherein the disorder is associated with propionbacterium acnes.

9. The composition of claim 1, wherein the disorder comprises cellulitis, cutaneous abscess, erysipelas, folliculitis, furuncles, impetigo, eethyma or necrotizing subcutaneous infection.

10. The composition of claim 1, wherein the disorder comprises acne or rosacea.

11. The composition of claim 1, wherein the disorder comprises syphilis, gonorrhea, chancroid, lymphogranuloma venereum, granuloma inguinale, a syndrome caused by chlamydia, mycoplasma, and ureaplasma infections, trichomoniasis or bacterial vaginosis.

12. A method of treating a disorder associated with a bacterial infection, consisting of administrating a pharmaceutical composition, comprising:
a. therapeutically effective amount of a conjugated boronic acid or a derivative thereof; and
b. a suitable pharmaceutical vehicle.

13. The method of claim 12, wherein the conjugated boronic acid comprises a compound wherein a boronic acid group is covalently linked with a moiety selected from the group consisting of:
a. a carbon atom which is further linked to an additional atom via a double bond, wherein the additional atom is selected from
i. A carbon atom
ii. A heteroatom
b. an aromatic ring
c. a polycyclic aromatic structure
d. a heterocyclic aromatic structure.

14. The method of claim 12, wherein the conjugated boronic acid comprises a formyl phenyl boronic acid.

15. The method of claim 12, wherein the conjugated boronic acid comprises formyl phenyl boronic acid is 2-formyl phenyl boronic acid.

16. The method of claim 12, wherein the form pharmaceutical composition is selected from the group consisting of:
a. a topical dosage form;
b. an enteral dosage form;
c. a parenteral dosage form;
d. a dosage form, suitable for epicutaneous, inhalation, rectal, ophthalmic, ear, intranasal or vaginal administration;
e. a dosage form comprising cream, gel, liniment, lotion, ointment, paste, spray, foam, mousse, lacquer or a transdermal patch;
f. a dosage form comprising a douche, an intrauterine device, a pessary, a vaginal ring, a vaginal tablet, enema, a rectal suppository, an aerosol inhaler, a metered dose inhalers, a solutions for nebulizer, eye drops, an ophthalmic gel or an ophthalmic ointment;
g. a dosage form comprising a buccal tablet, a sublingual tablet, a capsule, a suspension, a solution, a powder or drops;
h. a dosage form, suitable for injection or infusion
i. a dosage form, suitable for parenteral administration through a route selected from the group consisting of intravenous, intraarterial, intramuscular, intracardiac, subcutaneous; intraosseous infusion, intradermal, intrathecal and intraperitoneal.
j. a dosage form, suitable for parenteral administration through a route selected from the group consisting of transdermal, transmucosal, sublingual, buccal, vaginal, inhalational, epidural and intravitreal.

17. The method of claim 12, wherein the disorder is associated with bacteria comprising bacilli, cocci, spirochetes, gram-positive bacteria, gram-negative bacteria, aerobic bacteria or anaerobic bacteria.

18. The method of claim 12, wherein the disorder is associated with bacteria comprising a staphylococcus, a streptococcus, an enterococcus, Escherichia coli, Klebsiella, Escherichia coli, Enterobacter, Serratia or Pseudomonas aeruginosa.

19. The method of claim 17, wherein the disorder is associated with propionbacterium acnes.

20. The method of claim 12, wherein the disorder comprises cellulitis, cutaneous abscess, erysipelas, folliculitis, furuncles, impetigo, eethyma or necrotizing subcutaneous infection.

21. The method of claim 12, wherein the disorder comprises acne or rosacea.

22. The method of claim 12, wherein the disorder comprises syphilis, gonorrhea, chancroid, lymphogranuloma venereum, granuloma inguinale, a syndrome caused by chlamydia, mycoplasma, and ureaplasma infections, trichomoniasis or bacterial vaginosis.

23. A pharmaceutical composition, comprising:
c. a conjugated boronic acid or a derivative thereof; and
d. a pharmaceutical vehicle, selected from the group consisting of:
i. an oil in water emulsion
ii. an oil in water emulsion, comprising between 2% and 50% hydrophobic components
iii. an oil in water emulsion, comprising between 2% and 50% hydrophobic components, wherein the hydrophobic component is composed of at least two oils
iv. an oil in water emulsion, comprising between 2% and 50% hydrophobic components, wherein the hydrophobic component contains silicone
v. an oil in water emulsion, comprising between 2% and 50% hydrophobic components, wherein the oil component is composed of at least two oils, wherein the hydrophobic component further contains silicone
vi. an oil in water emulsion, wherein the surfactant used to stabilize the emulsion is a non-ionic surfactant
vii. an oil in water emulsion, wherein the surfactant used to stabilize the emulsion is a non-ionic surfactant having an HLB value of more than 9
viii. an oil in water emulsion, concurrently containing a non-ionic surfactant and a polymeric agent
ix. an oil in water emulsion, concurrently containing (i) a non-ionic surfactant, (ii) a polymeric agent; and (iii) an agent selected from a fatty alcohol and a fatty acid
x. a lacquer, suitable for application onto a keratinous surface
xi. a lacquer, simultaneously containing (i) a volatile solvent; and (ii) a polymeric agent
xii. a lacquer, simultaneously containing (i) a volatile solvent; and (ii) a film-forming polymeric agent
xiii. a water in oil emulsion
xiv. a water in oil emulsion, comprising between 20% and 80% hydrophobic components
xv. a water in oil emulsion, comprising between 20% and 80% hydrophobic components, wherein the hydrophobic component is composed of at least two oils
xvi. a water in oil emulsion, comprising between 20% and 80% hydrophobic components, wherein the hydrophobic component contains silicone
xvii. a water in oil emulsion, comprising between 20% and 80% hydrophobic components, wherein the oil component is composed of at least two oils, wherein the hydrophobic component further contains silicone
xviii. a water in oil emulsion, wherein the surfactant used to stabilize the emulsion is a non-ionic surfactant
xix. a water in oil emulsion, wherein the surfactant used to stabilize the emulsion is a non-ionic surfactant having an HLB value of more than 9
xx. a water in oil emulsion, concurrently containing a non-ionic surfactant and a polymeric agent
xxi. a water in oil emulsion, concurrently containing (i) a non-ionic surfactant, (ii) a polymeric agent; and (iii) an agent selected from a fatty alcohol and a fatty acid
xxii. a carrier, comprising at least 60% of a hydrophilic organic solvent
xxiii. a carrier, comprising at least 60% of a hydrophilic organic solvent, wherein the hydrophilic solvent is not volatile
xxiv. a carrier, comprising at least 60% of a hydrophilic organic solvent, wherein the hydrophilic solvent is not a lower alcohol
xxv. a carrier, comprising at least 60% of a hydrophilic organic solvent, wherein the hydrophilic organic solvent is selected from the group consisting of propylene glycol, polyethylene glycol and glycerin
xxvi. a carrier, comprising at least 60% of a hydrophilic organic solvent, wherein the hydrophilic organic solvent simultaneously contains at least two solvents, selected from the group consisting of propylene glycol, polyethylene glycol and glycerin
xxvii. a carrier, comprising at least 60% of a hydrophilic organic solvent, wherein the hydrophilic organic solvent simultaneously contains (i) at least two solvents, selected from the group consisting of propylene glycol, polyethylene glycol and glycerin, and (ii) a surfactant
xxviii. a carrier, comprising at least 60% of a hydrophilic organic solvent, wherein the hydrophilic organic solvent simultaneously contains (i) at least one solvent, selected from the group consisting of propylene glycol, polyethylene glycol and glycerin, (ii) a surfactant, and (iii) a polymeric agent
xxix. a carrier, comprising at least 60% of a hydrophilic organic solvent, wherein the hydrophilic organic solvent simultaneously contains (i) at least one hydrophilic solvent, selected from the group consisting of propylene glycol, polyethylene glycol and glycerin, (ii) a surfactant, (iii) a polymeric agent and (iv) an agent selected from a fatty alcohol and a fatty acid.
xxx. a carrier, comprising a hydrophilic organic solvent, wherein the carrier is substantially water-free
xxxi. a carrier, comprising at least 50% of petrolatum
xxxii. a carrier, comprising at least 50% of petrolatum, wherein the carrier is substantially water-free
xxxiii. a carrier, comprising at least 70% of petrolatum
xxxiv. a carrier, comprising at least 80% of petrolatum
xxxv. a carrier, simultaneously containing (i) at least 50% of petrolatum, and (ii) a surfactant
xxxvi. a carrier, simultaneously containing (i) at least 50% of petrolatum, and (ii) a surfactant, wherein the carrier is substantially water-free
xxxvii. a carrier, simultaneously containing (i) at least 50% of petrolatum, (ii) a surfactant and (iii) a polymeric agent and
xxxviii. a carrier, simultaneously containing (i) at least 50% of petrolatum, (ii) a surfactant, (iii) a polymeric agent and (iv) an agent selected from a fatty alcohol and a fatty acid.

24. A pharmaceutical foam composition, comprising any of the compositions provided in claim 23, provided that the composition contains components which are selected qualitatively and quantitatively to provide a foamable composition, wherein the foamable composition is packaged in an aerosol container and pressurized with a propellant.

25. The pharmaceutical composition of claim 24, wherein the propellant comprises a hydrocarbon propellant, a fluorine-containing propellant or a pressurized gas, in a suitable concentration to produce a foam.

26. Any of the pharmaceutical compositions of claim 23 or 24, wherein the conjugated boronic acid comprises phenyl boronic acid or a derivative thereof.

27. Any of the pharmaceutical compositions of claim 23 or 24, wherein the conjugated boronic acid comprises 2-formyl phenyl boronic acid or a derivative thereof.

28. Any of the pharmaceutical compositions of claim 23 or 24, wherein the carrier currier comprises
a. a dosage form, containing a pH-modifying system or a buffer system, suitable for maintaining the pH in the range between about 5.5 and about 9
b. a dosage form, containing a radical scavenger or an antioxidant
c. a dosage form, simultaneously containing (i) a pH-modifying system or a buffer system, suitable for maintaining the pH in the range between about 5.5 and about 9; and (ii) a radical scavenger or an antioxidant.

29. A pharmaceutical composition comprising:
a. a conjugated boronic acid or a derivative thereof;
b. a pharmaceutical vehicle suitable for treatment of a disorder associated with a bacterial infection.

30. The composition of claim 1 wherein the conjugated boronic acid is an unsubstituted or substituted phenyl group, having the general formula 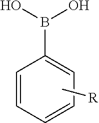
wherein the aromatic ring is linked to one OH group or to R, wherein R comprises H, alkyl, alkenyl, benzyl, CHO, OR′, NHR′, halogen, CONHR′ or COOR′; and R′ comprises H, alkyl, aryl, alkenyl, or benzyl.

31. The composition of claim 12 wherein the conjugated boronic acid is an unsubstituted or substituted phenyl group, having the general formula 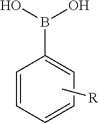
wherein the aromatic ring is linked to one OH group or to R, wherein R comprises H, alkyl, alkenyl, benzyl, CHO, OR′, NHR′, halogen, CONHR′ or COOR′; and R′ comprises H, alkyl, aryl, alkenyl, or benzyl.
